(19) United States
(12) Patent Application Publication
(10) Pub. No.: US 2002/0198500 A1
(43) Pub. Date: Dec. 26, 2002

(54) Non-reusable syringe

(76) Inventor: Valerie Leung, Georgetown (GY)

Correspondence Address: DINSMORE & SHOHL, LLP, 1900 CHEMED CENTER, 255 EAST FIFTH STREET, CINCINNATI, OH, 45202, US

(21) Appl. No.: 09/887,135

(22) Filed: Jun. 22, 2001

(51) Int. Cl. A61M 5/00

(52) U.S. Cl. 604/231; 604/110

(57) ABSTRACT

A non-reusable syringe. The syringe comprises a cylinder having a plunger, with the cylinder being adapted to receive a needle. The plunger has a piston rod with a piston at one end thereof and a handle at the opposed end, with the piston being within the cylinder and the handle extending from the cylinder. The piston is movable within the cylinder to permit filling of the cylinder with liquid. The cylinder has a section of reduced diameter in sliding engagement with the piston rod, the section of reduced diameter preventing removal of the piston from the cylinder. The piston has at least one orifice therethrough filled with a non-toxic substance that is dissolvable in the liquid.

### FIELD OF THE INVENTION

The present invention is directed to non-reusable syringes, and especially to such syringes in which the piston of the syringe cannot be removed from the cylinder of the syringe and which the piston is adapted for a single filling of the syringe with a liquid that is to be injected into a patient. In particular, the piston has an orifice therethrough that is filled with a non-toxic substance that will dissolve in the liquid, thereby preventing re-filling of the cylinder with liquid. In embodiments, the needle is also adapted for a single use e.g. the needle is retained in position by a substance that dissolves in the injectable liquid, thereby preventing re-use of the needle.

### BACKGROUND TO THE INVENTION

Syringes are used by a wide variety of health-care professional for the injection of liquids into patients. The liquids are typically medications, drugs, vaccines and a wide variety of other compositions that are intended to treat the patient against an infection, disease or other medical condition, or to prevent an infection or disease e.g. as in a vaccination. For hygienic reasons, such syringes and especially the needles of such syringes should only be used once.

Modern society includes a number of people who take drugs for recreational reasons, examples of such drugs being very numerous. Such recreational use of drugs frequently leads to addiction to drugs, and especially to more addictive and damaging drugs examples of which include heroin, cocaine. Drug addiction is a serious problem in many societies.

Drug addicts frequently share needles and have little regard for possible contamination of the needle, whether that contamination be for environmental, health or other reasons. Transmission of infectious diseases e.g. AIDS and hepatitis, is common amongst drug addicts, and the prime cause of the transmission of such disease is through shared use of needles. Thus, in addition to the devastating effects of the use of drugs per se, drug addicts often suffer from other debilitating diseases e.g. AIDS and hepatitis, that by themselves lead to premature death. Thus, drug addicts face two major health problems, use of the drugs per se and the infectious diseases that are a consequence of drug addiction.

Syringes are often of a type that are re-usable. Although medical professionals may not re-use a syringe, that syringe may be discarded with or without the needle attached. Some of the syringes and needles come into the possession of drug addicts, and are used repeatedly by the drug addicts.

Syringes that are intended for a single use are known. One such example is disclosed in WO 89/03231 of C. Sandsdalen, published Apr. 20, 1989. Such syringe has a piston that is formed from a material that will dissolve in the liquid to be injected into the patient, for instance crystalline glucose. The amount and type of crystalline glucose that is used is such that the syringe may be filled with liquid for use within a limited period of time e.g. two minutes, but repeated use is not possible due to dissolution of the glucose. Such a syringe would prevent a medical professional from re-using the syringe, but would be adaptable by a drug addict for further use.

### SUMMARY OF THE INVENTION

One aspect of the present invention provides a syringe comprising:

(a) a cylinder having a plunger, said cylinder being adapted to receive a needle;

(b) said plunger having a piston rod with a piston at one end thereof and a handle at the opposed end, said piston being within the cylinder and the handle extending from the cylinder, said piston being movable within the cylinder to permit filling of the cylinder with liquid;

said cylinder having a section of reduced diameter in sliding engagement with the piston rod, said section of reduced diameter preventing removal of the piston from the cylinder;

at least one of said piston and said cylinder having at least one orifice therethrough, said orifice being filled with a non-toxic substance that is dissolvable in the liquid.

In a preferred embodiment, said piston has at least one orifice therein, preferably two orifices or four orifices.

In another embodiment, said cylinder has at least one orifice therein.

In another preferred embodiment, the non-toxic substance is selected from the group consisting of glucose, sucrose, lactose, water-soluble polylactides, starch and dextrin.

In a further embodiment, the cylinder has an orifice therein juxtaposed to the needle.

Another aspect of the present invention provides a syringe comprising:

(a) a cylinder having a plunger, said cylinder being adapted to receive a needle;

(b) said plunger having a piston rod with a piston at one end thereof and a handle at the opposed end, said piston being within the cylinder and the handle extending from the cylinder, said piston being movable within the cylinder to permit filling of the cylinder with liquid;

said cylinder having a section of reduced diameter in sliding engagement with the piston rod, said section of reduced diameter preventing removal of the piston from the cylinder;

said piston having at least one orifice therethrough, said orifice being filled with a non-toxic substance that is dissolvable in the liquid.

### BRIEF DESCRIPTION OF THE DRAWINGS

The present invention is illustrated by the embodiments shown in the drawings, in which:

FIG. 1 is a schematic representation of a cross-section of an embodiment of the syringe of the present invention;

FIG. 2 is a schematic representation of a cross-section of a plunger of the syringe of FIG. 1;

FIG. 3 is a schematic representation of a cross-section of the piston of the plunger of FIG. 2, as viewed at A-A;

FIG. 4 is a schematic representation of an end view of an alternate embodiment of a syringe of the present invention.

### DETAILED DESCRIPTION OF THE INVENTION

The present invention relates to a syringe that is intended to be non-reusable. The syringe has a cylinder, or barrel, with a restriction to prevent removal of the piston of the plunger from the cylinder. In addition, the piston has orifices therein that are dissolved during use, thereby preventing re-use of the syringe.

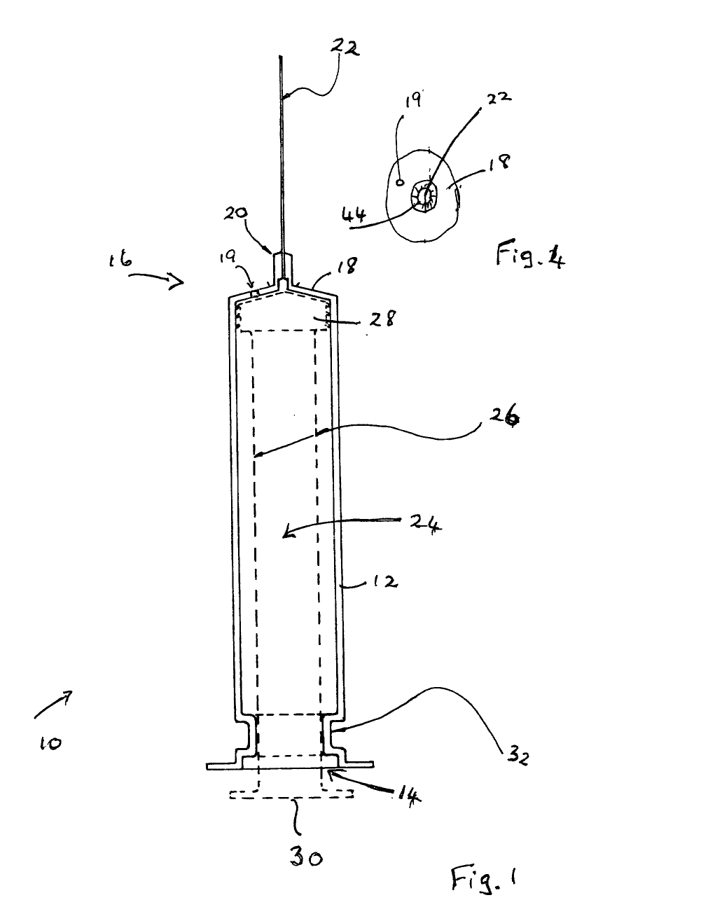

FIG. 1 shows a syringe of the present invention, generally indicated by 10. Syringe 10 is shown in cross-section. Syringe 10 has a cylinder 12 that is open at one end, 14. At the opposed or needle end, 16, cylinder 12 is tapered 18 and forms tip projection 20. In the embodiment shown, tip projection 20 has canula or needle 22 extending therefrom, needle 22 being permanently attached to tip projection 20. However, it is understood that tip projection 20 could be formed so as to accept a separate needle that has a base to fit over and be retained on tip projection 20. Needles adapted to fit onto syringes are known, as are caps or the like to cover the tip of the needle prior to use.

The embodiment of FIG. 1 is shown with cylinder 12 having cylinder orifice 19. Cylinder orifice 19 is optional, and if present would be filled with a material that will dissolve in the liquid that is drawn into the cylinder, as discussed below. Examples of such materials are given below.

Plunger 24 is located within cylinder 12. Plunger 24 has piston rod 26 that has piston 28 at one end and handle 30 at the opposed end. Plunger 24 is shown in greater detail in FIG. 2.

Juxtaposed to open end 14, cylinder 12 has constriction 32, which is a section of the cylinder that is of reduced diameter. Constriction 32 may be annular or partially annular. Constriction 32 acts as a guide for piston rod 26, and is in sliding engagement with piston rod 26 so as to effect smooth movement of plunger 24 within cylinder 12. In addition, constriction 32 prevents removal of plunger 24 from cylinder 12. As described herein, piston 28 of plunger 24 is of greater diameter than piston rod 26, being of the same diameter as cylinder 12 to facilitate normal functioning of the syringe for drawing of liquid into the syringe and injection of the liquid into a patient. Thus, piston 28 will not pass through constriction 32 of cylinder 12. It will be appreciated that plunger 24 must be inserted into cylinder 12 prior to constriction 32 being formed, as it is not possible to insert plunger 24 into cylinder 12 after constriction 32 is formed. Thus, cylinder 12 must be formed of a material, especially a plastic material that is capable of being re-shaped or constricted, by formation of the restriction, after plunger 24 is inserted. Similarly, plunger 24 and especially piston rod 26 thereof, must be formed of a material that is unaffected by the constriction of the cylinder, both in the sense of being reshaped and of absence of bonding to the cylinder during reshaping of the cylinder. It is understood that the constriction of the cylinder would normally be done using heat and pressure to form the restriction.

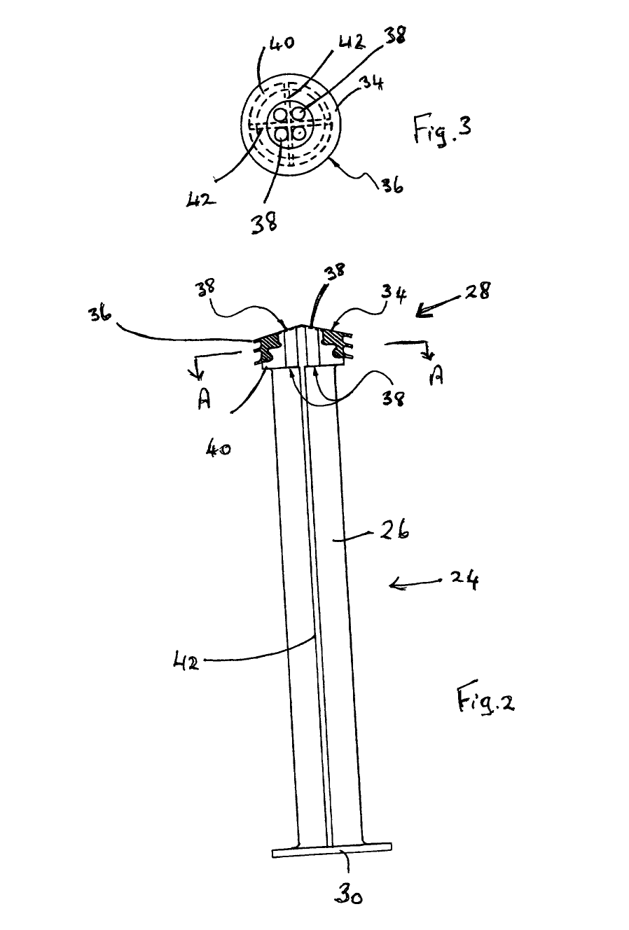

FIG. 2 shows plunger 24. Plunger 24 has piston rod 26 that extends between piston 28 and handle 30. Piston rod 24 may have a variety of cross-sectional shapes and is shown with a + cross-section, with ribs 42, which is a preferred cross-section for strength and ease of manufacture. Piston 28 has gasket 34 on piston head 40. Gasket 34 has squeegees 36 that form a fluid tight seal with the walls of cylinder 12 to prevent liquid from passing around piston 28 to the region of piston rod 26, as is known. Gasket 34 should conform to the internal shape of cylinder 12 at taper 18, as is known so that effectively all liquid in the syringe is injected into a patient during use. In addition, piston 28 has orifices 38 that extend therethrough, two such orifices being shown in the cross-section of FIG. 2. Piston 28 has at least one such orifice, and preferably at least two such orifices. Any convenient number of orifices may be used, and the view of FIG. 3 described below shows four orifices.

Orifices 38 are filled with a material that will dissolve in the liquid that is drawn into the syringe for injection into a patient. It is understood that dimensions of the orifice, especially diameter and length, and the material used to fill the orifices need to be selected so that a user has sufficient time to fill the syringe with the liquid to be injected into a patient and to complete the injection in to a patient prior to the material in the orifice dissolving and forming an open passage through piston 28. That period of time should be 2-5 minutes, although the time may be varied. It will be appreciated that any amounts of liquid remaining in the syringe after use, including small amounts, will continue to effect dissolution of the material after use of the syringe. The material used to fill the orifices must also be non-toxic to the patient.

Examples of materials that may be used include glucose, sucrose, lactose, water-soluble polylactides, starch and dextrin. In an embodiment, a mesh e.g. a nylon mesh, may be inserted into the orifice, with the mesh being impregnated with the material that will dissolve. This provides physical strength to the material within the orifice, while still permitting dissolution.

FIG. 3 shows a cross-section of piston 28, through A-A of FIG. 2. Piston 28 has gasket 34, with squeegee edge 36, on piston head 40. Piston 28 has four orifices 38 therein that are filled with the dissolvable material. The + cross-section of piston rod 26, with ribs 42 is also shown.

FIG. 4 shows an end view of one embodiment of cylinder 12 in which needle 22 is embedded in taper 18 by means of a dissolvable material 44. In this embodiment, dissolution of dissolvable material 44 would further disable the syringe, making the syringe more difficult to re-use. The end view also shows the optional embodiment of cylinder orifice 19.

In use, liquid is drawn into the syringe, and then injected into a patient with a limited period of time. As discussed above, that period of time is substantially pre-determined by selection of the dimensions of the orifices and the material within the orifices. After use, the orifices are open, and it becomes impossible to refill the syringe with any liquid. It would also be very difficult to inject a substance into orifices within the piston of the syringe without plugging the syringe so that is was not usable. Moreover, the plunger cannot be removed from the syringe to effect plugging of the orifices. The embodiment of FIG. 4 makes it more difficult to render the syringe re-usable as a needle would have to be re-set into the syringe.

The present invention has been described herein with reference to plunger 24 having orifices therein filled with the dissolvable material and with reference to cylinder 12 having orifices therein filled with dissolvable material as illustrated in FIG. 1 and FIG. 4. The syringe of the invention has at least one of such orifices. In particularly preferred embodiments, the syringe has the orifices in the plunger, and optionally has at least one of the types of orifices shown in FIGS. 1 and 4.

The present invention provides a non-reusable syringe or alternatively a syringe that is extremely difficult to return to a usable condition. Thus, the invention provides a syringe that essentially cannot be re-used by health care professionals, drug addicts or other persons, and thus is not susceptible for transmission of infectious diseases between persons receiving injections.

1. A syringe comprising:
(a) a cylinder having a plunger, said cylinder being adapted to receive a needle;
(b) said plunger having a piston rod with a piston at one end thereof and a handle at the opposed end, said piston being within the cylinder and the handle extending from the cylinder, said piston being movable within the cylinder to permit filling of the cylinder with liquid;
said cylinder having a section of reduced diameter in sliding engagement with the piston rod, said section of reduced diameter preventing removal of the piston from the cylinder;
at least one of said piston and said cylinder having at least one orifice therethrough, said orifice being filled with a non-toxic substance that is dissolvable in the liquid.

2. The syringe of claim 1 in which said piston has at least one orifice therein.

3. The syringe of claim 1 in which said cylinder has at least one orifice therein.

4. The syringe of claim 2 in which said cylinder has at least one orifice therein.

5. The non-reusable syringe of claim 1 in which the non-toxic substance is selected from the group consisting of glucose, sucrose, lactose, water-soluble polylactides, starch and dextrin.

6. The non-reusable syringe of claim 2 in which the non-toxic substance is selected from the group consisting of glucose, sucrose, lactose, water-soluble polylactides, starch and dextrin.

7. The non-reusable syringe of claim 2 in which the piston has two orifices.

8. The non-reusable syringe of claim 2 in which the piston has four orifices.

9. The non-reusable syringe of claim 3 in which the cylinder has an orifice therein juxtaposed to the needle.

10. A syringe comprising:
(a) a cylinder having a plunger, said cylinder being adapted to receive a needle;
(b) said plunger having a piston rod with a piston at one end thereof and a handle at the opposed end, said piston being within the cylinder and the handle extending from the cylinder, said piston being movable within the cylinder to permit filling of the cylinder with liquid;
said cylinder having a section of reduced diameter in sliding engagement with the piston rod, said section of reduced diameter preventing removal of the piston from the cylinder;
said piston having at least one orifice therethrough, said orifice being filled with a non-toxic substance that is dissolvable in the liquid.

11. The non-reusable syringe of claim 10 in which the non-toxic substance is selected from the group consisting of glucose, sucrose, lactose, water-soluble polylactides, starch and dextrin.

12. The non-reusable syringe of claim 10 in which the piston has two orifices.

13. The non-reusable syringe of claim 10 in which the piston has four orifices.
